(19) United States
(12) Patent Application Publication
(10) Pub. No.: US 2012/0141498 A1
(43) Pub. Date: Jun. 7, 2012

(54) USE OF PKC ISOZYMES FOR DIAGNOSIS AND TREATMENT OF NEUROBLASTOMA

(75) Inventors: Mildred E. Acevedo-Duncan, Plant City, FL (US); Rekha S. Patel, Tampa, FL (US)

(73) Assignees: United States Government as Represented by the Department of Veterans Affairs, Washington, DC (US); University of South Florida, Tampa, FL (US)

(21) Appl. No.: 13/310,376

(22) Filed: Dec. 2, 2011

Related U.S. Application Data
(60) Provisional application No. 61/419,006, filed on Dec. 2, 2010.
(60) Provisional application No. 61/515,131, filed on Aug. 4, 2011.

(51) Int. Cl. A61K 39/395; A61K 31/713; A61P 35/00; A61K 38/45; C12Q 1/48; G01N 33/573; A61K 48/00

(52) U.S. Cl. 424/158.1; 435/7.4; 514/44 A; 514/44 R; 424/94.5; 435/15

(57) ABSTRACT

The present invention pertains to the use of PKC-epsilon (PKC-ε), PKC-delta (PKC-δ), PKC-eta, and/or PKC-theta as biomarker(s) for prediction and/or detection of neuroblastoma, as well as therapeutic targets for treatment of neuroblastoma.

### CROSS-REFERENCE TO RELATED APPLICATIONS

This application claims the benefit of U.S. Provisional Application Ser. Nos. 61/419,006, filed Dec. 2, 2010, and 61/515,131, filed Aug. 4, 2011, which are hereby incorporated by reference in their entirety.

### FIELD OF INVENTION

This invention relates to use of PKC-epsilon, PKC-delta, PKC-eta, and PKC-theta for diagnosis and treatment of neuroblastoma.

### BACKGROUND

Neuroblastomas (neuro: nerve and blastoma: cancer) are highly lethal tumors that originate in the sympathetic nervous system: the network of nerves transmitting neuronal messages originating in the brain to various parts of the body. Neuroblastoma has its primary site as the adrenal glands; however, it may also occur in other tissues like abdomen, pelvis, neck or spinal cord. Neuroblastoma is the fourth most common type of cancer in children and most commonly occurs in infants. According to the American Cancer Society, there are approximately 650 new cases of neuroblastoma each year in the United States.

Protein kinase C (PKC) is a family of fourteen known isozymes which are found in varying ratios in the cytoplasmic and membrane fraction of cells depending on the type of tissue and its physiological state. PKC isozymes can be classified into three groups. Group I includes Ca²⁺ dependent isozymes: cPKC-α, cPKC-β_{I}, cPKC-β_{II}, and cPKC-γ. Isozymes in group II (nPKC-ε, nPKC-δ, nPKC-η and PKC-θ) are Ca²⁺ independent. Group III includes the atypical PKCs (aPKC-ι, aPKC-ζ, PKMζ (a brain-specific isoform of PKC-zeta generated from an alternative transcript), aPKC-μ (protein kinase D) and aPKC-ν), which are insensitive to both diacylglycerol and calcium and neither bind to nor are activated by phorbol esters.

PKC regulates cellular functions, metabolism and proliferation by phosphorylating proteins in response to transmembrane signals from hormones, growth factors, neuro-transmitters, and pharmacological agents. Activation of PKC by various agonists (including radiation) results in altered transcription of a considerable number of genes. Some PKC isozymes are transiently translocated from the cytosol to a membrane structure. Membrane association leads to binding alterations in PKC's regulatory subunit (phospholipid-/diacylglycerol/phorbol ester) and its 50 KD catalytic domain (ATP/substrate). For PKCs to be activated, phosphoinositide-dependent kinase (PDK-1) docks on the carboxyl terminus of unphosphorylated PKC, PDK-1 phosphorylates PKCs on the activation loop, and upon release of PDK-1, the carboxyl terminus is unmasked and allows autophosphorylation. This sequence of phosphorylation events is required before PKCs are able to respond to cofactor second messengers (phosphatidylserine/diacylglycerol). Proteolytic degradation of membrane PKC leads to its down-regulation. PKC is the major receptor for tumor promoting phorbol esters, but the extent of PKC involvement in cellular malignancy is not clearly defined.

Despite significant educational efforts, improved diagnostic techniques, and rigorous therapies, neuroblastoma control remains static. Certain neuroblastomas are highly lethal tumors due to the emergence of therapy-resistant neuroblastoma cells. Previous work has shown that PKCs are involved in the proliferation of neuroblastoma cells in culture; however, it is well known that in-vitro cell culture experiments may not represent the physiological environment in vivo.

### BRIEF SUMMARY

The present invention pertains to the use of PKC-epsilon (PKC-ε), PKC-delta (PKC-δ), PKC-eta (PKC-η), and/or PKC-theta (PKC-θ) as biomarker(s) for prediction and/or detection of neuroblastoma, as well as therapeutic targets for treatment of neuroblastoma.

In one embodiment, the present invention provides a method for predicting whether a subject is at risk of developing neuroblastoma, or already has neuroblastoma, comprising:

(a) obtaining a biological sample from a subject;

(b) determining in the sample a level of expression for one or more PKC isozymes selected from the group consisting of PKC-epsilon, PKC-delta, PKC-eta, and PKC-theta; and

(c) comparing the expression level in (b) to a level of expression in a normal control,

wherein if a level of expression of PKC-epsilon and/or PKC-delta is determined, overexpression of PKC-epsilon and/or PKC-delta, with respect to the control, indicates that the subject is at risk of developing neuroblastoma; and

wherein if a level of expression of PKC-eta and/or PKC-theta is determined, decreased level of expression of PKC-eta and/or PKC-theta, with respect to the control, indicates that the subject is at risk of developing, or already has, neuroblastoma.

In another aspect, the present invention provides methods for treatment of neuroblastoma. In one embodiment, the method comprises administering, to a subject in need of such treatment, an effective amount of a therapeutic agent selected from:

a) an inhibitor of PKC-epsilon and an inhibitor of PKC-delta; and

b) PKC-eta, PKC-theta, a nucleic acid molecule for expression of PKC-eta, a nucleic acid molecule for expression of PKC-theta, an agent that increases expression of PKC-eta, an agent that increases expression of PKC-theta, a mimetic of PKC-eta, and a mimetic of PKC-theta.

In another aspect, the present invention provides assays for screening for inhibitors of PKC-epsilon and/or PCK-delta as well as mimetics of PKC-eta and/or PKC-theta as candidate therapeutics for treatment of neuroblastoma.

In one embodiment, the present invention provides a method for screening for PKC-epsilon and/or PCK-delta inhibitors as candidate therapeutics for treatment of neuroblastoma, comprising:

providing an agent that inhibits PKC-epsilon, PCK-delta, or both;

contacting neuroblastoma cells with the agent;

determining whether growth or proliferation of the neuroblastoma cells is slowed; and, if so,

identifying the agent as a candidate therapeutic agent for treatment of neuroblastoma.

In another embodiment, the present invention provides a method for screening for a mimetic of PKC-eta and/or PCK-theta as candidate therapeutics for treatment of neuroblastoma, comprising:

providing an agent that is a mimetic of PKC-eta or PCK-theta;

contacting neuroblastoma cells with the agent;

determining whether growth or proliferation of the neuroblastoma cells is slowed; and, if so,

identifying the agent as a candidate therapeutic for treatment of neuroblastoma.

### BRIEF DESCRIPTION OF THE SEQUENCES

SEQ ID NO: 1 is an amino acid sequence of human protein kinase C-epsilon (GenBank Accession No. CAA46388.1).

SEQ ID NO: 2 is an amino acid sequence of human protein kinase C-delta (GenBank Accession No. NP_{—}006245.2).

SEQ ID NO: 3 is an amino acid sequence of human protein kinase C-eta (GenBank Accession No. NP_{—}006246).

SEQ ID NO:4 is an amino acid sequence of human protein kinase C-theta (GenBank Accession No. NP_{—}006248).

### BRIEF DESCRIPTION OF THE DRAWINGS

FIG. 1 shows expression of PKC-α, -δ and -ε in normal adrenal, normal kidney, and neuroblastoma tissue. Normal adrenal, kidney, and neuroblastoma tissue biopsies (80 μg) are subjected to gel electophoresis. Western blotting is performed with antibodies against PKC-ε (BD Transduction, San Diego, Calif.); PKC-δ (Santa Cruz Biotechnology); and PKC-α (Santa Cruz). Secondary antibodies are obtained from Accurate JOM035146, Westbury, N.Y.) and used at 1.5:10000 dilution (48 μg). Immunoblots from 7 normal adrenal specimens (1N-7N), 7 normal kidney (8N-14N) and 12 neuroblastomas (1T-12T) are used for comparison. The tumors specimens are classified as follows: 1T, metastatic neuroblastoma unspecified; 2T, kidney neuroblastoma; 3T, right chest neuroblastoma; 4T, surrounding bone neuroblastoma; 5T, abdominal neuroblastoma, 6T, metastatic abdominal neuroblastoma; 7T, adrenal neuroblastoma; 8T, abdominal mass neuroblastoma; 9T, abdominal lymphoid, 10T, artery neuroblastoma; 11T, neuroblastoma unspecified; 12T neuroblastoma unspecified.

FIG. 2 shows PKC-η and PKC-θ expression in normal adrenal, normal kidney, and neuroblastoma tissue. Normal adrenal (1N-7N), normal kidney (1N-8N), adrenal neuroblastoma (1T-7T), and kidney neuroblastoma (1T-6T) tissue specimens are purchased from Nationwide Children's Hospital Biopathology Center (Columbus, Ohio). Tissue biopsies (80 μg) are subjected to gel electrophoresis. Western blots for PKC-η and PKC-θ are performed using tissue lysates with either monoclonal antibodies against PKC-η (cat. # P64720, BD Transduction, San Diego, Calif.) at a 1:1000 dilution (5 μg) or monoclonal antibodies against PKC-θ (cat. #610090, BD Transduction, San Diego, Calif.) at a 1:1000 dilution (5 μg). Secondary antibodies are obtained from Accurate JOM035146, Westbury, N.Y.) and used at 1.5:10000 dilution (2.5 μg). The results show that PKC-η, which is expressed in most of normal adrenal and kidney tissue, is not expressed in adrenal as well as kidney neuroblastoma tissue. The results also show that PKC-θ, which is expressed in most of normal kidney tissue, is not expressed in kidney neuroblastoma tissue.

### DETAILED DESCRIPTION

The present invention pertains to the use of PKC-epsilon (PKC-ε), PKC-delta (PKC-δ), PKC-eta (PKC-η), and/or PKC-theta (PKC-θ) as biomarker(s) for prediction and/or detection of neuroblastoma, as well as therapeutic targets for treatment of neuroblastoma. The invention is based on the discovery that PKC-epsilon and PKC-delta, which are not expressed or are minimally expressed in normal tissue, are significantly overexpressed in neuroblastoma. As shown in the Examples, PKC-ε is found to be overexpressed in 11 out of 12 neuroblastomas (including kidney and adrenal neuroblastomas), when compared to normal adrenal and kidney. In contrast, no PKC-ε or PKC-δ is detected in normal adrenal or kidney. PKC-δ is overexpressed in 8 out of 12 neuroblastomas, but is not found in kidney or adrenal neuroblastomas. In addition, there is a loss of PKC-eta (PKC-η) and PKC-theta (PKC-θ) expression in neuroblastoma.

### Prediction and Diagnosis of Neuroblastoma

One aspect of the present invention pertains to the use of PKC-epsilon, PKC-delta, PKC-eta, and/or PKC-theta as biomarker(s) for prediction and/or detection of neuroblastoma.

In one embodiment, the present invention provides a method for predicting whether a subject is at risk of developing neuroblastoma, comprising:

(a) obtaining a biological sample from a subject;

(b) determining in the sample a level of expression for one or more PKC isozymes selected from the group consisting of PKC-epsilon, PKC-delta, PKC-eta, and PKC-theta; and

(c) comparing the expression level in (b) to a level of expression in a normal control, wherein if a level of expression of PKC-epsilon and/or PKC-delta is determined, overexpression of PKC-epsilon and/or PKC-delta, with respect to the control, indicates that the subject is at risk of developing neuroblastoma;

wherein if a level of expression of PKC-eta and/or PKC-theta is determined, decreased level of expression of PKC-eta and/or PKC-theta, with respect to the control, indicates that the subject is at risk of developing neuroblastoma.

In another embodiment, the present invention provides a method for diagnosing whether a subject has neuroblastoma, comprising:

(a) obtaining a biological sample from a subject;

(b) determining in the sample a level of expression for one or more PKC isozymes selected from the group consisting of PKC-epsilon, PKC-delta, PKC-eta, and PKC-theta; and

(c) comparing the expression level in (b) to a level of expression in a normal control, wherein if a level of expression of PKC-epsilon and/or PKC-delta is determined, overexpression of PKC-epsilon and/or PKC-delta, with respect to the control, indicates that the subject has neuroblastoma;

wherein if a level of expression of PKC-eta and/or PKC-theta is determined, decreased level of expression of PKC-eta and/or PKC-theta, with respect to the control, indicates that the subject has neuroblastoma.

In a further embodiment, the presence or absence of PKC-epsilon and/or PKC-delta is determined, wherein the presence of PKC-epsilon, or PKC-delta, or both, indicates that the subject has, or is at risk of developing, neuroblastoma.

In another further embodiment, the presence or absence of PKC-eta and/or PKC-theta is determined, wherein if the sample contains no detectable PKC-epsilon, or PKC-delta, or both, indicates that the subject has, or is at risk of developing, neuroblastoma.

In another further embodiment, the expression levels of PKC-epsilon, PKC-delta, PKC-eta, and PKC-theta are measured, wherein overexpression of PKC-epsilon and PKC-delta as well as decreased levels of expression of PKC-eta and PKC-theta, with respect to the control, indicates that the subject has, or is at risk of developing, neuroblastoma.

In a further embodiment, decreased level of expression of PKC-theta, with respect to the control, indicates that the subject has, or is at risk of developing, kidney neuroblastoma. In another embodiment, decreased level of expression of PKC-eta, with respect to the control, indicates that the subject has, or is at risk of developing, adrenal and/or kidney neuroblastoma.

The term “subject,” as used herein, describes an organism, including mammals such as primates, to which treatment with the compositions according to the subject invention can be administered. Mammalian species that can benefit from the disclosed methods include, but are not limited to, apes, chimpanzees, orangutans, humans, monkeys; and other animals such as dogs, cats, horses, cattle, pigs, sheep, goats, chickens, mice, rats, guinea pigs, and hamsters. Typically, the subject is a human.

The term “biological sample,” as used herein, includes but is not limited to, a sample containing tissues, cells, and/or biological fluids isolated from a subject. Examples of biological samples include but, are not limited to, tissues, cells, bodily fluids, biopsies, blood, lymph, serum, plasma, urine, saliva, and tears. In preferred embodiments, the biological sample is a blood, urine, tissue, or a bodily fluid sample. The tissue or bodily fluid samples can include cells or tissues of adrenal, kidney, bone marrow, bone(s), abdominal, lymph, chest, spinal cord, pelvis, neck, and cells and/or tissues of other locations where neuroblastoma can potentially form. In a specific embodiment, the biological sample is a tissue biopsy sample or a bone marrow aspiration sample. In another embodiment, the biological sample comprises nerve tissue or cells. In another embodiment, the biological sample is obtained from a cyst, lump, polyp, or tumor that is suspected as a neuroblastoma.

In one embodiment, the control level of PKC isozyme expression is determined by measuring expression level of a PKC isozyme of interest (such as PKC-epsilon, PKC-delta, PKC-eta, and PKC-theta) in a healthy population that do not have neuroblastoma.

The level of PKC isozyme expression can be determined based on mRNA levels or protein levels. Determination of PKC isozyme expression can be made qualitatively, semi-quantitatively, or quantitatively. Sequences of various PKC isozyme proteins and mRNAs of a variety of animal species are publicly available and can be obtained from, for example, the GenBank database. For instance, human PKC-epsilon protein has an amino acid sequence of SEQ ID NO:1; human PKC-delta protein has an amino acid sequence of SEQ ID NO:2; human PKC-eta protein has an amino acid sequence of SEQ ID NO:3; and human PKC-theta protein has an amino acid sequence of SEQ ID NO:4. One of ordinary skill in the art, having the benefit of the present disclosures, can easily use publicly-available PKC-epsilon, -delta, -eta, and -theta sequences of an animal species (including human) of interest to practice the present invention.

Methods for determining PKC isozyme expression levels are well known in the art, including but not limited to, Western blots, Northern blots, Southern blots, enzyme-linked immunosorbent assay (ELISA), immunoprecipitation, immunofluorescence, radioimmunoassay, flow cytometry, immunocytochemistry, nucleic acid hybridization techniques, nucleic acid reverse transcription methods, nucleic acid amplification methods, and any combination thereof.

In one embodiment, the level of PKC protein expression is determined by contacting the biological sample with a binding agent, such as an antibody, or aptamer, that specifically recognizes, or specifically binds to, a PKC isozyme protein of interest (such as PKC-epsilon, -delta, -eta, and -theta); and detecting the complex between the binding agent and the PKC isozyme protein. In preferred embodiments, a binding agent, such as an antibody, or aptamer, specific to a PKC isozyme of interest does not recognize or bind to any other PKC isozymes. For instance, antibody specific to PKC-epsilon does not recognize or bind to any other PKC isozyme that is not PKC-epsilon. In certain embodiments, the level of PKC isozyme expression can be determined by immunoassays including, but not limited to, radioimmunoassay, Western blot assay, ELISA, immunofluorescent assay, enzyme immunoassay, immunoprecipitation, chemiluminescent assay, immunohistochemical assay, dot blot assay, and slot blot assay.

A contacting step in the assay (method) of the invention can involve contacting, combining, or mixing the biological sample and the solid support, such as a reaction vessel, microvessel, tube, microtube, well, multi-well plate, or other solid support.

Samples and/or agents that specifically bind to a PKC isozyme of interest may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or “physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or 1536-well plates (or trays). Optionally, agents that specifically bind to a PKC isozyme of interest (such as PKC-epsilon, -delta, -eta, and -theta) may be immobilized on the solid support.

In a further embodiment, the diagnostic assay of the present invention is used in combination with other diagnostic or screening test for neuroblastoma, such as physical exams, cytogenetic analysis, X rays, CT scan, neurological exam, biopsy, bone marrow aspiration, ultrasound, and MIBC (metaiodobenzylguanidine) scan. In addition, the presence of certain physical symptoms may aid the detection of neuroblastoma. Symptoms that may suggest the development of neuroblastoma include lump in the abdomen, neck, or chest; bulging eyes; bone pain; swollen stomach and trouble breathing in infants; painless, bluish lumps under the skin in infants; weakness or paralysis.

In another aspect, the present invention includes kits comprising the required elements for detecting PKC-epsilon, -delta, -eta, and/or -theta. Preferably, the kits comprise a container for collecting a sample, and an agent for detecting the presence of PKC-epsilon, -delta, -eta, and/or -theta in the sample. The components of the kits can be packaged either in aqueous medium or in lyophilized form.

The methods of the invention can be carried out using a diagnostic kit for qualitatively or quantitatively detecting PKC-epsilon, -delta, -eta, and/or -theta in a sample. By way of example, the kit can contain binding agents (for example, antibodies or aptamers) specific for PKC-epsilon, -delta, -eta, and/or -theta, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit.

As indicated above, kits of the invention include reagents for use in the methods described herein, in one or more containers. The kits may include specific internal controls, and/or probes, buffers, and/or excipients, separately or in combination. Each reagent can be supplied in a solid form or liquid buffer that is suitable for inventory storage. Kits may also include means for obtaining a sample from a host organism or an environmental sample.

Kits of the invention can be provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like. Preferably, the solid matrix is a structure having a surface that can be derivatized to anchor an oligonucleotide probe, primer, molecular beacon, specific internal control, etc. Preferably, the solid matrix is a planar material such as the side of a microtiter well or the side of a dipstick. In certain embodiments, the kit includes a microtiter tray with two or more wells and with reagents including primers, probes, specific internal controls, and/or molecular beacons in the wells.

Kits of the invention may optionally include a set of instructions in printed or electronic (e.g., magnetic or optical disk) form, relating information regarding the components of the kits and/or how to make various determinations (e.g., PKC-epsilon, -delta, -eta, and/or -theta levels, comparison to control standards, etc.). The kit may also be commercialized as part of a larger package that includes instrumentation for measuring other biochemical components.

### Treatment of Neuroblastoma

In another aspect, the present invention provides methods for treatment of neuroblastoma. In one embodiment, the method comprises administering, to a subject in need of such treatment, an effective amount of a therapeutic agent selected from:

a) an inhibitor of PKC-epsilon and an inhibitor of PKC-delta; and

b) PKC-eta, PKC-theta, a nucleic acid molecule for expression of PKC-eta, a nucleic acid molecule for expression of PKC-theta, an agent that increases expression of PKC-eta, an agent that increases expression of PKC-theta, a mimetic of PKC-eta, and a mimetic of PKC-theta.

In one embodiment, the present invention administers agents that specifically inhibit PKC-epsilon, or PKC-delta, or both, but do not substantially inhibit any other PKC isozyme or isoform that is neither PKC-epsilon nor PKC-delta. In another embodiment, the present invention administers agents that increase expression of PKC-eta, PKC-theta, or both, but do not increase the expression of any other PKC isozyme or isoform that is neither PKC-eta nor PKC-theta. In a specific embodiment, the present invention does not administer agents that increase the expression of PKC-epsilon or PKC-delta. In one embodiment, the PKC-eta or PKC-theta mimetic is not a mimetic of any other PKC isozyme that is neither PKC-eta nor PKC-theta.

The term “treatment” or any grammatical variation thereof (e.g., treat, treating, and treatment etc.), as used herein, includes but is not limited to, ameliorating or alleviating a symptom of a disease or condition; reducing or delaying recurrence of a condition; reducing, suppressing, inhibiting, lessening, or affecting the progression and/or severity of an undesired physiological change or a diseased condition. For instance, treatment includes, for example, preventing, inhibiting, or slowing rate of formation of neuroblastoma; slowing the growth and/or proliferation of neuroblastoma; inhibiting metastasis of neuroblastoma.

The term “effective amount,” as used herein, refers to an amount that is capable of treating or ameliorating a disease or condition or otherwise is capable of producing an intended therapeutic effect. In certain embodiments, the effective amount enables a 5%, 10%, 20%, 30%, 40%, 50%, 75%, 90%, 95%, 99% or 100% reduction in the size of neuroblastoma.

In an embodiment, a subject in need of the treatment of the present invention has, or is diagnosed of having, neuroblastoma. In another embodiment, a subject in need of the treatment of the present invention is at risk of having neuroblastoma. In another embodiment, a subject in need of the treatment of the present invention has neuroblastoma as well as an overexpression of PKC-epsilon and/or PKC-delta, when compared to that of a normal population that do not have neuroblastoma. In another embodiment, a subject in need of the treatment of the present invention has neuroblastoma as well as decreased expression of PKC-eta and/or PKC-theta, when compared to that of a normal population that do not have neuroblastoma. In an embodiment, the therapeutic agent is delivered to the neuroblastoma tissue.

In an embodiment, the present invention provides a method for treating neuroblastoma. In certain specific embodiments, the present invention can be used to treat neuroblastoma selected from kidney, adrenal, chest, bone, abdominal, and/or artery neuroblastoma. In certain specific embodiments, the present invention provides the use of inhibitor of PKC-epsilon and/or PKC-delta for treatment of neuroblastoma selected from kidney, adrenal, chest, bone, abdominal, and/or artery neuroblastoma. In another specific embodiment, the present invention provides the use of PKC-eta, an agent that increases expression of PKC-eta, or a mimetic of PKC-eta for treatment of kidney and adrenal neuroblastoma. In another specific embodiment, the present invention provides the use of PKC-theta, an agent that increases expression of PKC-theta, or a mimetic of PKC-theta for treatment of kidney neuroblastoma. The present invention can be used to treat primary, localized neuroblastoma and/or metastatic neuroblastoma.

In an embodiment, the present invention excludes the administration of PKC inhibitors that also inhibit the expression and/or activity of a PKC isozyme that is not PKC-epsilon or PKC-delta including, but not limited to, antibodies, binding partners, and/or aptamers that bind to a PKC protein isozyme that is not PKC-epsilon or PKC-delta; antisense nucleic acid molecules that inhibit the expression of a PKC protein isozyme that is not PKC-epsilon or PKC-delta; and/or compounds (such as chelerythrine chloride) that inhibit a PKC protein isozyme that is not PKC-epsilon or PKC-delta. In a specific embodiment, the present invention excludes the administration of an agent that inhibits PKC-eta or PKC-theta.

In an embodiment, the present invention excludes the administration of a PKC isozyme that is not PKC-eta or PKC-theta; an agent that increases expression of a PKC isozyme that is not PKC-eta or PKC-theta; and/or a mimetic of a PKC-isozyme that is not PKC-eta or PKC-theta. In a specific embodiment, the present invention excludes the administration of PKC-epsilon or PKC-delta, an agent that increases activity and/or expression of PKC-epsilon or PKC-delta, and mimetic of PKC-epsilon or PKC-delta.

### PKC-epsilon and PKC-delta Inhibitors

The present invention pertains to uses of PKC-epsilon and PKC-delta inhibitors for treatment of neuroblastoma. Inhibitors of PKC-epsilon and PKC-delta useful according to the present invention include, but are not limited to, agents that inhibit activity of PKC-epsilon and PKC-delta, respectively; and agents that reduce or inhibit the expression of PKC-epsilon and PKC-delta, such as agents that inhibit the transcription, translation, and/or processing of PKC-epsilon and PKC-delta, respectively.

Agents that inhibit PKC-epsilon and PKC-delta activity include, but are not limited to, anti-PKC-epsilon and anti-PKC-delta antibodies, aptamers, PKC-epsilon and PKC-delta binding partners, and small molecule inhibitors of PKC-epsilon and PKC-delta, respectively. In one embodiment, an inhibitor of PKC-epsilon or PKC-delta is an antibody that binds specifically to PKC-epsilon or PKC-delta. In a further specific embodiment, an inhibitor of PKC-epsilon or PKC-delta is an antibody that binds specifically to human PKC-epsilon or PKC-delta. In some embodiments, inhibitors of PKC-epsilon or PKC-delta include PKC-epsilon or PKC-delta antibodies that bind specifically to PKC-epsilon or PKC-delta proteins of non-human animals including, but not limited to, apes, chimpanzees, orangutans, monkeys, dogs, cats, horses, pigs, sheep, goats, mice, rats, and guinea pigs. The skilled artisan could easily construct PKC-epsilon- or PKC-delta-specific antibodies to specifically target any PKC-epsilon or PKC-delta proteins publicly known. In a specific embodiment, the PKC-epsilon inhibitor is an antibody that binds specifically to a human PKC-epsilon of SEQ ID NO:1. In another specific embodiment, the PKC-delta inhibitor is an antibody that binds specifically to a human PKC-delta of SEQ ID NO:2.

“Specific binding” or “specificity” refers to the ability of a protein to detectably bind an epitope presented on a protein or polypeptide molecule of interest, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific target molecule versus nonspecific binding to other irrelevant molecules.

Anti-PKC-epsilon and anti-PKC-delta antibodies of the present invention can be in any of a variety of forms, including intact immunoglobulin molecules, fragments of immunoglobulin molecules such as Fv, Fab and similar fragments; multimers of immunoglobulin molecules (e.g., diabodies, triabodies, and bi-specific and tri-specific antibodies, as are known in the art; see, e.g., Hudson and Kortt, J. Immunol. Methods 231:177-189, 1999); fusion constructs containing an antibody or antibody fragment; and human or humanized immunoglobulin molecules or fragments thereof.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Antibodies of the present invention include polyclonal and monoclonal antibodies. The term “monoclonal antibody,” as used herein, refers to an antibody or antibody fragment obtained from a substantially homogeneous population of antibodies or antibody fragments (i.e. the individual antibodies within the population are identical except for possible naturally occurring mutations that may be present in a small subset of the antibody molecules).

A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 1975, 256:495-497, the disclosure of which is herein incorporated by reference. An exemplary hybridoma technology is described by Niman et al., Proc. Natl. Acad. Sci. U.S.A., 1983, 80:4949-4953. Other methods of producing monoclonal antibodies, a hybridoma cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoise as described by Sasatry, et al., Proc. Natl. Acad. Sci. USA, 1989, 86:5728-5732; and Huse et al., Science, 1981, 246:1275-1281. The references cited are hereby incorporated herein by reference.

In one embodiment of the invention, monoclonal antibodies specific for PKC-epsilon and PKC-delta can be used as a delivery vehicle for drug or toxin. Drug or toxin can be conjugated to the antibodies using a biochemical approach. Monoclonal antibodies specific for the amino-terminus of PKC-epsilon or PKC-delta can be used as a delivery vehicle for drug or toxin. This enables the transport of drug or toxin to tumor cells with high expression of PKC-epsilon and PKC-delta.

There are many known PKC-epsilon inhibitors. Embodiments of inhibitors of PKC-epsilon are described in, for example, U.S. Patent Application Publication No. 2009/0124533; U.S. Patent Application Publication No. 2009/0318351; U.S. Pat. Nos. 6,376,467, 6,686,334, 5,783,405, 5,141,957, 5,204,370, 5,216,014, and 5,432,198 which are hereby incorporated by reference. There are also many known PKC-delta inhibitors. Peptide inhibitors of PKC-delta are described in, for example, U.S. Patent Application Publication No. 2009/0155236, which is hereby incorporated by reference. A person skilled in the art, having benefit of the present invention, can select suitable inhibitors of PKC-epsilon and PKC-delta for treatment of neuroblastoma.

In some embodiments, PKC-epsilon and PKC-delta inhibitors useful according to the present invention are agents that reduce or inhibit the expression of PKC-epsilon and PKC-delta, respectively, such as agents that inhibit the transcription, translation, and/or processing of PKC-epsilon and PKC-delta.

In an embodiment, the PKC-epsilon or PKC-delta inhibitor is a PKC-epsilon or PKC-delta antisense polynucleotide. In an embodiment, the PKC-epsilon or PKC-delta inhibitor is an antisense polynucleotide that targets human PKC-epsilon or PKC-delta mRNA. In some embodiments, the PKC-epsilon or PKC-delta antisense polynucleotides target PKC-epsilon or PKC-delta mRNAs of non-human animals including, but not limited to, apes, chimpanzees, orangutans, monkeys, dogs, cats, horses, pigs, sheep, goats, mice, rats, and guinea pigs. The skilled artisan would readily appreciate that the antisense polynucleotides can be designed to target any PKC-epsilon and PKC-delta mRNAs publicly known.

In some embodiments, the PKC-epsilon or PKC-delta inhibitor is a siRNA having a sequence sufficiently complementary to a target PKC-epsilon or PKC-delta mRNA sequence to direct target-specific RNA interference (RNAi). In some embodiments, the PKC-epsilon or PKC-delta inhibitor is siRNA having a sequence sufficiently complementary to a target human PKC-epsilon or PKC-delta mRNA sequence (such as mRNA encoding SEQ ID NO:1 or SEQ ID NO:2) to direct target-specific RNA interference.

Examples of antisense polynucleotides include, but are not limited to, single-stranded DNAs and RNAs that bind to complementary target PKC-epsilon or PKC-delta mRNA and inhibit translation and/or induce RNaseH-mediated degradation of the target transcript; siRNA oligonucleotides that target or mediate PKC-epsilon or PKC-delta mRNA degradation; ribozymes that cleave PKC-epsilon or PKC-delta mRNA transcripts; and nucleic acid aptamers and decoys, which are non-naturally occurring oligonucleotides that bind to and block PKC-epsilon or PKC-delta protein targets in a manner analogous to small molecule drugs.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog”, also referred to herein as an “altered nucleotide” or “modified nucleotide,” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of endogenous target genes, such as PKC-epsilon and PKC-delta.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a siRNA having a “sequence sufficiently complementary to a target mRNA sequence to direct target-specific RNA interference (RNAi)” means that the siRNA has a sequence sufficient to trigger the destruction of the target mRNA (e.g., PKC-epsilon or PKC-delta mRNA) by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

The present invention also contemplates vectors (e.g., viral vectors) and expression constructs comprising the nucleic acid molecules useful for inhibiting PKC-epsilon or PKC-deltaexpression and/or activity. In an embodiment, the vector comprises a siRNA that targets PKC-epsilon or PKC-delta mRNA. In another embodiment, the vector comprises a nucleic acid molecule encoding an anti-PKC-epsilon or PKC-delta antibody.

As used herein, the term “expression construct” refers to a combination of nucleic acid sequences that provides for transcription of an operably linked nucleic acid sequence. As used herein, the term “operably linked” refers to a juxtaposition of the components described, wherein the components are in a relationship that permits them to function in their intended manner. In general, operably linked components are in contiguous relation.

Expression constructs of the invention will also generally include regulatory elements that are functional in the intended host cell in which the expression construct is to be expressed. Thus, a person of ordinary skill in the art can select regulatory elements for use in, for example, bacterial host cells, yeast host cells, mammalian host cells, and human host cells. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

### Screening Assays

In another aspect, the present invention provides assays for screening for inhibitors of PKC-epsilon and/or PCK-delta as well as mimetics of PKC-eta and/or PKC-theta as useful candidate therapeutics for treatment of neuroblastoma.

In one embodiment, the present invention provides a method for screening for PKC-epsilon and/or PCK-delta inhibitors as candidate therapeutics for treatment of neuroblastoma, comprising:

providing an agent that inhibits PKC-epsilon, PCK-delta, or both;

contacting neuroblastoma cells with the agent;

determining whether growth or proliferation of the neuroblastoma cells is slowed; and, if so,

identifying the agent as a candidate therapeutic agent for treatment of neuroblastoma.

In another embodiment, the present invention provides a method for screening for a mimetic of PKC-eta and/or PCK-theta as candidate therapeutics for treatment of neuroblastoma, comprising:

providing an agent that is a mimetic of PKC-eta or PCK-theta;

contacting neuroblastoma cells with the agent;

determining whether growth or proliferation of the neuroblastoma cells is slowed; and, if so,

identifying the agent as a candidate therapeutic agent for treatment of neuroblastoma.

Neuroblastoma cells for use in the present screening assays can be selected from, for example, kidney, adrenal, chest, bone, abdominal, artery neuroblastoma cells, or neuroblastoma; and non-metastatic and metastatic neuroblastoma cells.

### Therapeutic Compositions and Formulations

The present invention further provides therapeutic compositions that contain an effective amount of a therapeutic agent and a pharmaceutically acceptable carrier or adjuvant.

The therapeutic agent can be formulated in a variety of forms. These include for example, solid, semi-solid, and liquid dosage forms, such as tablets, pills, powders, liquid solutions or suspensions, suppositories, and injectable and infusible solutions. The preferred form depends on the intended mode of administration and therapeutic application.

In a preferred embodiment, the composition is formulated in accordance with routine procedures as a pharmaceutical composition adapted for local injection administration to human beings. Typically, compositions for local injection administration are solutions in sterile isotonic aqueous buffer. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

The present invention also provides for a therapeutic method by administering therapeutic or pharmaceutical compositions in a form that can be combined with a pharmaceutically acceptable carrier. In this context, the compound may be, for example, isolated or substantially pure. The term “carrier” refers to a diluent, adjuvant, excipient, or vehicle with which the compound is administered. Such pharmaceutical carriers can be sterile liquids, such as water and oils, including those of petroleum oil such as mineral oil; vegetable oil such as peanut oil, soybean oil, and sesame oil; animal oil; or oil of synthetic origin.

Suitable carriers also include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic composition, if desired, can also contain minor amounts of wetting or emulsifying agents, or pH buffering agents.

The amount of active ingredient that may be combined with the carrier materials to produce a single dosage form will vary, depending on the type of the condition and the subject to be treated. The amount of active ingredient that may be combined with the carrier materials to produce a single dosage form will vary, depending on the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

The therapeutic agents of the invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the present invention.

The therapeutic or pharmaceutical compositions of the present invention can also be formulated as neutral or salt forms. Pharmaceutically acceptable salts include, but are not limited to, hydrochloric, phosphoric, acetic, oxalic, sodium, potassium, ammonium, calcium, ferric hydroxides, isopropylamine, and triethylamine salts.

### Routes of Administration

The therapeutic agents and compositions of the present invention can be administered to the subject being treated by standard routes, including oral, or parenteral administration including intravenous, intramuscular, and intraspinal injection, infusion, and electroporation, as well as co-administration as a component of any medical device or object to be inserted (temporarily or permanently) into a subject.

In some embodiments, the methods disclosed herein include contacting a neuroblastoma or tumor cells with an effective amount of a therapeutic agent.

The amount of the therapeutic or pharmaceutical composition of the present invention effective in the treatment of neuroblastoma will depend on a variety of factors, such as the route of administration and the seriousness of the condition, and should be decided according to the judgment of the practitioner and each patient's circumstances. In general, the dosage ranges from about 0.01 μg/kg to about 10 mg/kg, about 0.01 μg/kg to about 1 mg/kg, about 0.01 μg/kg to about 100 μg/kg, about 0.01 μg/kg to about 10 μg/kg, or about 0.01 μg/kg to about 1 μg/kg. Such a unit dose may be administered once to several times (e.g. two, three and four times) every two weeks, every week, or every day.

In one embodiment, the therapeutic agents and compositions of the present invention and any second therapeutic agent are administered simultaneously or sequentially to the patient, with the second therapeutic agent being administered before, after, or both before and after treatment with the compounds of the present invention. Sequential administration may involve treatment with the second therapeutic agent on the same day (within 24 hours) of treatment with the subject compound. Sequential administration may also involve continued treatment with the second therapeutic agent on days that the subject compound is not administered.

### EXAMPLES

Following are examples that illustrate embodiments for practicing the invention. These examples should not be construed as limiting.

### Example 1

### Overexpression of PKC-epsilon and PKC-delta in Neuroblastoma

Patient-derived normal and malignant adrenal tissue specimens are purchased from the Collaborative Tissue Network and obtained from the All Children's Hospital. Tissue specimens are obtained from children of varying ages. Tumors were classified as follows: 1T, metastatic neuroblastoma unspecified; 2T, kidney neuroblastoma; 3T, right chest neuroblastoma; 4T, surrounding bone neuroblastoma; 5T, abdominal neuroblastoma, 6T, metastatic abdominal neuroblastoma; 7T, adrenal neuroblastoma; 8T, abdominal mass neuroblastoma; 9T, abdominal lymphoid, 10T, artery neuroblastoma; 11T, neuroblastoma unspecified; 12T neuroblastoma unspecified.

Normal adrenal, kidney and neuroblastoma tissue biopsies (80 μg) are subjected to gel electophoresis. Western blotting is performed with antibodies against PKC-ε (BD Transduction, San Diego, Calif.); PKC-δ (Santa Cruz Biotechnology); and PKC-α (Santa Cruz). Secondary antibodies are obtained from Accurate JOM035146, Westbury, N.Y.) and used at 1.5:10000 dilution (48 μg). Immunoblots from 7 normal adrenal specimens (1N-7N), 7 normal kidney (8N-14N) and 12 neuroblastomas (1T-12T) are used for comparison.

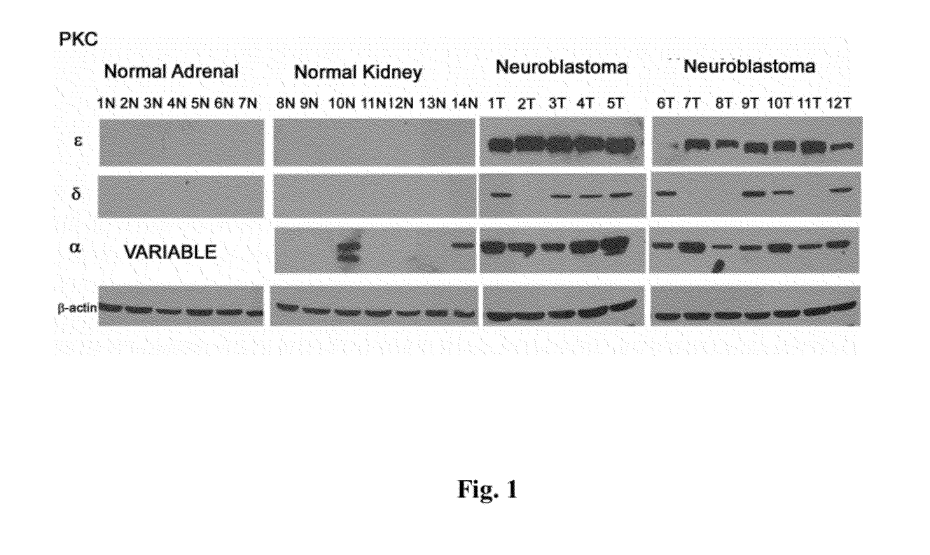

As shown in FIG. 1, PKC-ε is overexpressed in 11 out of 12 neuroblastomas (including kidney and adrenal neuroblastoma tissue), when compared to normal adrenal and kidney tissue. No PKC-ε or PKC-δ is detected in normal adrenal or kidney. PKC-δ in overexpressed in 8 out of 12 neuroblastomas, but is not detected in kidney or adrenal neuroblastomas. PKC-α has variable expression in the normal adrenal and is present in 2 out of 7 normal kidneys. PKC-α is detected in all the neuroblastomas, including the kidney neuroblastoma.

The results show that PKC-ε and PKC-δ can be used as a biomarker for detection of neuroblastoma, as well as therapeutic targets for the treatment of neuroblastoma.

### Example 2

### Loss of PKC-eta and PKC-theta Expression in Neuroblastoma

Normal adrenal (1N-7N), normal kidney (1N-8N), adrenal neuroblastoma (1T-7T), and kidney neuroblastoma (1T-6T) tissue specimens are purchased from Nationwide Children's Hospital Biopathology Center (Columbus, Ohio). Tissue biopsies (80 μg) are subjected to gel electrophoresis. Western blots for PKC-η and PKC-θ are performed using tissue lysates with either monoclonal antibodies against PKC-η (cat. # P64720, BD Transduction, San Diego, Calif.) at a 1:1000 dilution (5 μg) or monoclonal antibodies against PKC-θ (cat. #610090, BD Transduction, San Diego, Calif.) at a 1:1000 dilution (5 μg). Secondary antibodies are obtained from Accurate JOM035146, Westbury, N.Y.) and used at 1.5:10000 dilution (2.5 μg).

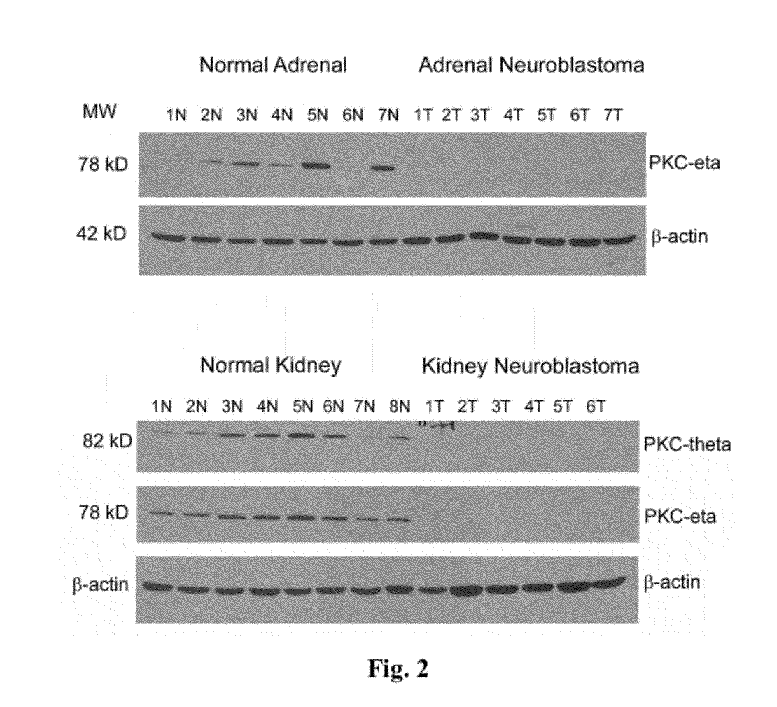

As shown in FIG. 2, PKC-η, which is expressed in 6/7 (86%) of normal adrenal and all of normal kidney tissue, is absent in adrenal and kidney neuroblastoma (100%). The level of PKC-θ expression varies in the normal adrenal, is absent in ⅞ (87%) of the normal kidney. PKC-θ expression is lost in all (6/6) kidney neuroblastoma (FIG. 2).

The results show that PKC-η as well as PKC-θ acts as a repressor of malignant progression. Specifically, the loss of PKC-η contributes to both adrenal and kidney neuroblastoma, while the loss of PKC-θ contributes to kidney neuroblastoma. This indicates that therapeutic agents that act as mimetics of PKC-η and PKC-θ can be used to inhibit or delay malignant progression. Thus, PKC-η and PKC-θ can be used as therapeutic targets for the treatment of neuroblastoma. The results also show that loss of PKC-η and/or PKC-θ can be used as a biomarker to detect neuroblastoma or predict whether the subject is at risk of developing neuroblastoma.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A method for determining whether a subject has neuroblastoma, or is at risk of developing neuroblastoma, comprising:
(a) obtaining a biological sample from a subject;
(b) determining in the sample a level of expression for one or more PKC isozymes selected from the group consisting of PKC-epsilon, PKC-delta, PKC-eta, and PKC-theta; and
(c) comparing the expression level in (b) to a level of expression in a normal control,
wherein if a level of expression of PKC-epsilon and/or PKC-delta is determined, overexpression of PKC-epsilon and/or PKC-delta, with respect to the control, indicates that the subject has or is at risk of developing neuroblastoma; and
wherein if a level of expression of PKC-eta and/or PKC-theta is determined, decreased level of expression of PKC-eta and/or PKC-theta, with respect to the control, indicates that the subject has or is at risk of developing neuroblastoma.

2. The method according to claim 1, wherein a level of expression of PKC-epsilon and/or PKC-delta is determined, wherein overexpression of PKC-epsilon, PKC-delta, or both, with respect to the control, indicates that the subject has or is at risk of developing neuroblastoma.

3. The method according to claim 1, wherein a level of expression of PKC-eta and/or PKC-theta is determined, wherein decreased level of expression of PKC-eta, PKC-theta, or both, with respect to the control, indicates that the subject has or is at risk of developing neuroblastoma.

4. The method according to claim 1, wherein the subject is a human.

5. The method according to claim 1, wherein the biological sample is selected from blood, urine, or biopsy sample.

6. The method according to claim 1, wherein the comparing step includes: contacting the sample with an antibody that specifically recognizes the PKC isozyme protein of which the expression level is to be determined; and detecting the complex between the antibody and said PKC isozyme protein.

7. The method according to claim 6, wherein said PKC isozyme protein is contacted with an antibody that specifically recognizes said PKC isozyme in an immunoassay selected from the group consisting of radioimmunoassay, western blot assay, immunofluorescent assay, enzyme immunoassay, immunoprecipitation, chemiluminescent assay, immunohistochemical assay, dot blot assay, and slot blot assay.

8. A method for treating neuroblastoma comprising administering, to a subject in need of such treatment, an effective amount of a therapeutic agent selected from:
a) an inhibitor of PKC-epsilon or an inhibitor of PKC-delta; and
b) PKC-eta, PKC-theta, a nucleic acid molecule for expression of PKC-eta, or a nucleic acid molecule for expression of PKC-theta;
whereby the neuroblastoma is treated.

9. The method according to claim 8, wherein the therapeutic agent is an inhibitor of PKC-epsilon or an inhibitor of PKC-delta.

10. The method according to claim 9, wherein the therapeutic agent is a PKC-epsilon-specific small interfering RNA molecule or a PKC-delta-specific small interfering RNA molecule.

11. The method according to claim 9, wherein the therapeutic agent is a PKC-epsilon-specific antibody or a PKC-delta-specific antibody.

12. The method according to claim 8, wherein the therapeutic agent is selected from PKC-eta, PKC-theta, a nucleic acid molecule for expression of PKC-eta, or a nucleic acid molecule for expression of PKC-theta.

13. A method for screening for PKC-epsilon and/or PCK-delta inhibitors as candidate therapeutics for treatment of neuroblastoma, comprising:
providing an agent that inhibits PKC-epsilon, PCK-delta, or both;
contacting neuroblastoma cells with the agent;
determining whether growth or proliferation of the neuroblastoma cells is slowed; and, if so,
identifying the agent as a candidate therapeutic for treatment of neuroblastoma.

14. A method for screening for a mimetic of PKC-eta and/or PCK-theta as a candidate therapeutic for treatment of neuroblastoma, comprising:
providing an agent that is a mimetic of PKC-eta or PCK-theta;
contacting neuroblastoma cells with the agent;
determining whether growth or proliferation of the neuroblastoma cells is slowed; and, if so,
identifying the agent as a candidate therapeutic agent for treatment of neuroblastoma.


SEQUENCE LISTING (4 sequences)

SEQ ID NO: 1 - protein, 737 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDARGIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 2 - protein, 676 aa, Homo sapiens
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 3 - protein, 683 aa, homosapien
MSSGTMKFNGYLRVRIGEAVGLQPTRWSLRHSLFKKGHQLLDPYLTVSVDQVRVGQTSTKQKTNKPTYNEEFCANVTDGGHLELAVFHETPLGYDHFVANCTLQFQELLRTTGASDTFEGWVDLEPEGKVFVVITLTGSFTEATLQRDRIFKHFTRKRQRAMRRRVHQINGHKFMATYLRQPTYCSHCREFIWGVFGKQGYQCQVCTCVVHKRCHHLIVTACTCQNNINKVDSKIAEQRFGINIPHKFSIHNYKVPTFCDHCGSLLWGIMRQGLQCKICKMNVHIRCQANVAPNCGVNAVELAKTLAGMGLQPGNISPTSKLVSRSTLRRQGKESSKEGNGIGVNSSNRLGIDNFEFIRVLGKGSFGKVMLARVKETGDLYAVKVLKKDVILQDDDVECTMTEKRILSLARNHPFLTQLFCCFQTPDRLFFVMEFVNGGDLMFHIQKSRRFDEARARFYAAEIISALMFLHDKGIIYRDLKLDNVLLDHEGHCKLADFGMCKEGICNGVTTATFCGTPDYIAPEILQEMLYGPAVDWWAMGVLLYEMLCGHAPFEAENEDDLFEAILNDEVVYPTWLHEDATGILKSFMTKNPTMRLGSLTQGGEHAILRHPFFKEIDWAQLNHRQIEPPFRPRIKSREDVSNFDPDFIKEEPVLTPIDEGHLPMINQDEFRNFSYVSPELQP

SEQ ID NO: 4 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS